(19) United States
(12) Patent Application Publication
(10) Pub. No.: US 2009/0325147 A1
(43) Pub. Date: Dec. 31, 2009

(54) MOLECULARLY IMPRINTED POLYMERS FOR DETECTING MICROORGANISMS

(75) Inventor: Robert L. Jones, JR., Columbia, MD (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Columbia BioSystems, Inc., Baltimore, MD (US)

(21) Appl. No.: 12/492,857

(22) Filed: Jun. 26, 2009

Related U.S. Application Data
(60) Provisional application No. 61/076,353, filed on Jun. 27, 2008.
(60) Provisional application No. 61/169,450, filed on Apr. 15, 2009.

(51) Int. Cl. C12Q 1/70; G01N 33/53; G01N 33/554; C12Q 1/02; C08F 301/00

(52) U.S. Cl. 435/5; 435/7.2; 435/7.32; 435/29; 526/72

(57) ABSTRACT

The invention described herein provides molecularly imprinted polymers (MIPs) that are capable of binding to a microorganism, and methods for detecting and/or identifying microorganisms utilizing Molecularly Imprinted Polymers (MIPs). The microorganisms of the invention include prokaryotes, eukaryotes, virus and prions. The methods of the invention comprise detecting all or part, including epitopes, of macromolecules associated with the microorganisms. The macromolecules of the invention include polysaccharides, proteins, glycoproteins, peptidoglycans, lipoproteins, peptides, polypeptides, and polynucleotides, associated with said microorganisms. The invention also provides for methods of diagnosing a subject infected with the microorganisms utilizing MIPs, in addition to diagnostic kits.

### CROSS REFERENCE TO RELATED APPLICATION

The present application claims priority to U.S. Provisional Application No. 61/076,353, filed on Jun. 27, 2008, and 61/169,450, filed on Apr. 15, 2009, the disclosures of both of which are specifically incorporated herein by reference in their entirety.

### FIELD OF THE INVENTION

The invention described herein generally relates to molecularly imprinted polymers (MIPs) that are capable of binding to microorganisms, including methods and kits for detecting, identifying and/or quantifying microorganisms utilizing MIPs.

### BACKGROUND OF THE INVENTION

MIPs are engineered cross-linked polymers that exhibit high affinity and selectivity towards a single compound or a family of related compounds. MIPs are able to bind analytes even when these are present in complex matrices (e.g., plasma, urine, muscle tissue, food matrices, environmental samples, process solutions etc). An important strength of MIPs is that they are able to bind to trace levels of target molecule, in the presence of large excess of other compounds that have similar physico-chemical properties. Unlike most separation particles that exhibit only non-selective interactions, MIPs have a selective synthetic recognition site (or imprint), which is sterically and chemically complementary to a particular target or class of targets. MIPs are economical and fast to produce and are robust and stable under storage. They can be used at elevated temperatures, in organic solvents and at extreme pH values. They also display a higher sample load capacity than is typical for immunoaffinity based sorbents. This results in higher recoveries for analytical applications and suitability of using the sorbents for semi-preparative or preparative scale separations.

Molecular imprinting involves arranging polymerizable functional monomers around a template (for example, a pseudo-target molecule, an analog of the target molecule, all or portion of the actual target molecule, etc) followed by polymerization and template removal. The arrangement is typically achieved by: (i) non-covalent interactions (e.g., H-bonds, ion pair interactions) or (ii) reversible covalent interactions. After template removal, these molecularly imprinted polymers can recognize and bind to the actual target molecule.

MIPs hold several advantages over antibodies for diagnostics and sample analysis, due to their controlled synthesis and remarkable stability. Molecular imprinting originates from the concept of creating tailor-made recognition sites in polymers by template polymerization (Mosbach K. et al., Bio/Technology, 1996, 14, 163-170; Ansell R. J. et al., Curr. Opin. Biotechnol., 1996, 7, 89-94; Wulff G. Angew. Chem. Int. Ed. Engl., 1995, 34, 1812-32; Vidyasankar S. et al., Curr. Opin. Biotechnol., 1995, 6, 218-224; and Shea K. J, Trends In Polymer Science, 1994, 2, 166-173). Molecularly imprinted polymers demonstrated remarkable recognition properties that were applied in various fields such as drug separation (Fischer L., et al., J. Am. Chem. Soc, 1991, 113, 9358- 9360; Kempe M, et al., J. Chromatogr., 1994, 664, 276-279; Nilsson K., et al., J. Chromatogr., 1994, 680, 57-61), receptor mimics (Ramstrom O., et al., Tetrahedron: Asymmetry, 1994, 5, 649-656; Ramstrom O., et al., J. Mol. Recogn., 1996, 9, 691-696; Andersson L. L, et al., Proc. Natl. Acad. Sci., 1995, 92, 4788-4792; Andersson L. L, Anal. Chem., 1996, 68, 111-117), bio-mimetic sensors (Kriz D., et al., Anal. Chem., 1995, 67, 2142-2144), antibody mimics (Vlatakis G., et al., Nature, 1993, 361, 645- 647), template-assisted synthesis (Bystrom S. E., et al, J. Am. Chem. Soc, 1993, 115, 2081-2083), and catalysis (Muller R., et al., Makromol. Chem., 1993, 14, 637-641; Beach J. V., et al., J. Am. Chem. Soc, 1994, Vol. 116, 379-380).

The great potential embodied in MIPs resulted in numerous inventions for analytical devices and methods of detection of various targets, based on molecular imprinting, reviewed by Ye and Haupt (Anal. Bioanal. Chem. 2004, 378,1887-1897). Some examples of MIP-based sensors are described in U.S. Pat. Nos 5,587,273, 6,680,210, 6,833,274, 6,967,103 and 6,461,873. Using MIPs combined with displacement of analyte-marker conjugate was shown to be practical in several laboratories (Vlatakis G. et al., Nature, 1993, 361, 645-647, Levi et al., 1997, Anal. Chem. 69. 2017-2021; Nathaniel T. et al., J. Am. Chem. Soc. 2005, 127, 5695-5700; Nicholls C. et al, Biosens. Bioelec, 2006, 21, 1171-1177).

To date, molecular imprints have had limited application to the binding of larger molecules including macromolecules. Synthetic polymers which selectively bind amino acid derivatives and peptides were created using the target amino acid derivative or peptide as a template (Kemp, 1996, Anal. Chem. 68:1948-1953). Imprints have also been created which bind to nucleotide derivatives (Spivak and Shea, 1998, Macromolecules 31:2160-2165). Ionic molecular images of polypeptides have been created by mixing a matrix material with the intact polypeptide chain to be bound by the molecular image (U.S. Pat. No. 5,756,717). Molecular imprints of cytochrome c, hemoglobin and myoglobin, respectively, have been prepared by polymerizing acrylamide in the presence of each intact protein. An imprint of horse myoglobin selectively bound horse myoglobin from a mixture of proteins including whale myoglobin (U.S. Pat. No. 5,814,223).

Although the methods of molecular imprinting have shown limited success at selectively binding macromolecules, the methods have not been utilized to detect or identify microorganisms. These shortcomings in the art are overcome by the invention described below, which in one aspect provides MIPs useful for detecting, identifying and/or quantifying microorganisms in a sample or on a target area. Generally, the imprint compositions of the invention described below comprise a matrix material defining an imprint of a template microorganism. Potential advantages of MIP-based materials include: specificity comparable to a biorecognition element; robustness and stability under extreme chemical and physical conditions; and an ability to design recognition sites for target molecules that lack suitable biorecognition elements.

### SUMMARY OF THE INVENTION

One embodiment of the invention described herein provides molecularly imprinted polymers (MIPs) capable of specifically binding to a microorganism.

Another embodiment of the invention provides methods of detecting and/or identifying a microorganism, comprising contacting one or more MIPs with said microorganism.

Yet another embodiment of the invention provides methods for diagnosing a subject infected with a microorganism, comprising contacting a biological sample obtained from said subject with one or more MIPs, and detecting and/or identifying the presence of said microorganism in said biological sample.

In one embodiment of the invention, the microorganism can be selected from the group consisting of prokaryotes, eukaryotes, virus and prions.

In one embodiment, the MIPs are capable of binding to all or a portion of a macromolecule unique to said microorganism.

In another embodiment of the invention, the MIPs are capable of binding to an epitope of said macromolecule.

In one embodiment of the invention, the macromolecules can be selected from the group consisting of exopolysaccharides, polysaccharides, proteins, glycoproteins, peptidoglycans, lipoproteins, peptides, polypeptides, and polynucleotides.

In one embodiment of the invention, the MIPs are capable of binding to all or a portion of a macromolecule associated with Methicillin-Resistant S. aureus (MRSA).

In one embodiment of the invention, the macromolecule associated with MRSA is penicillin binding protein 2a (PBP2a).

In another embodiment of the invention, the MIPs are capable of binding to amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and/or fragments thereof.

In one embodiment of the invention, the MIPs are coupled with transduction elements such that a measurable signal is produced in response to binding of MIPs to said microorganism.

In one embodiment of the invention, the MIPs are coupled with transduction elements such that a measurable signal is produced in response to binding of MIPs to all or a portion, of said macromolecule.

In one embodiment of the invention, the signal associated with the transduction element is selected from the group consisting of colorimetric, fluorescence, radioactive and enzymatic.

In one embodiment of the invention, the biological sample is selected from the group consisting of biological fluids, tissue extracts and tissues.

In one embodiment of the invention, the biological fluid can be selected from the group consisting of blood, cerebrospinal fluid, serum, plasma, urine, nipple aspirate, fine needle aspirate, tissue lavage, saliva, sputum, ascites fluid, semen, lymph, vaginal pool, synovial fluid, spinal fluid, amniotic fluid, breast milk, pulmonary sputum or surfactant, urine, fecal matter, fluids collected from any of liver, kidney, breast, bone, bone marrow, testes, brain, ovary, skin, lung, prostate, thyroid, pancreas, cervix, stomach, intestine, colorectal, bladder, colon, nares and uterine, head and neck, nasopharynx tumors, and other liquid samples of biologic origin.

In one embodiment of the invention, the tissues can be selected from the group consisting of liver, kidney, breast, testes, brain, ovary, skin, head and neck, lung, prostate, thyroid, pancreas, cervix, stomach, intestine, colorectal, bladder, colon and uterine.

In one embodiment of the invention, the tissue extracts can be selected from the group consisting of liver, kidney, breast, testes, brain, ovary, skin, head and neck, lung, prostate, thyroid, pancreas, cervix, stomach, intestine, colorectal, bladder, colon and uterine extracts.

One embodiment of the invention provides for a method of diagnosing a patient infected with a microorganism, comprising contacting a biological sample obtained from said patient with one or more MIPs, and detecting and/or identifying the presence of a macromolecule unique to said microorganism in said biological sample.

One embodiment of the invention provides for a method of detecting or identifying MRSA utilizing MIPs that are capable of binding to all or a portion of a macromolecule associated with MRSA.

Another embodiment of the invention provides for a method of detecting or identifying MRSA utilizing MIPs that are capable of binding to all or portion of PBP2a.

Another embodiment of the invention provides for a method of detecting or identifying MRSA utilizing MIPs that are capable of binding to amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and/or fragments thereof.

In one embodiment of the invention, a detectable signal is produced upon binding of all or a portion of a macromolecule associated with MRSA.

In one embodiment of the invention, a detectable signal is produced upon binding of MRSA with MIPs that are capable of binding to all or a portion of PBP2a.

In another embodiment of the invention, a detectable signal is produced upon the binding of MRSA with MIPS that are capable of binding to amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and/or fragments thereof.

One embodiment of the invention provides for methods of diagnosing a patient with MRSA infection.

In one embodiment of the invention, the method of diagnosing a patient with MRSA infection comprises contacting a biological sample obtained from said patient with one or more MIPs that are capable of binding to a macromolecule associated with MRSA.

In another embodiment of the invention, the method of diagnosing a patient with MRSA infection comprises measuring the detectable signal produced upon binding of MRSA in the biological sample obtained from said patient to MIPs that are capable of binding to a macromolecule associated with MRSA.

In one embodiment of the invention, the method of diagnosing a patient with MRSA infection comprises contacting a biological sample obtained from said patient with one or more MIPs that are capable of binding to all or portion of PBP2a.

In another embodiment of the invention, the method of diagnosing a patient with MRSA infection comprises measuring the detectable signal produced upon binding of MRSA in the biological sample obtained from said patient to MIPs that are capable of binding to all or portion of PBP2a.

In one embodiment of the invention, the method of diagnosing a patient with MRSA infection comprises contacting a biological sample obtained from said patient with one or more MIPs that are capable of binding to amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and/or fragments thereof.

In another embodiment of the invention, the method of diagnosing a patient with MRSA infection comprises measuring the detectable signal produced upon binding of MRSA in the biological sample obtained from said patient to MIPs that are capable of binding to amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and/or fragments thereof.

One embodiment of the invention provides methods for determining the onset, progression, or regression of an infection associated with a microorganism in a subject, wherein a biological sample obtained from a subject is screened for said microorganism by contacting said biological sample with one or more MIPs.

One embodiment of the invention provides a kit comprising one or more MIPs for detecting or identifying a microorganism.

Another embodiment of the invention provides methods to detect the presence of microorganisms on a target area, comprising contacting the target area with one or more MIPs.

In one embodiment, the target area includes environmental surfaces, such as in hospitals, sports equipment and medical devices.

In another embodiment of the invention, the target area can be selected from the group consisting of bed railing, door knobs and computer keyboards.

### BRIEF DESCRIPTION OF THE DRAWINGS

FIG. 1 illustrates a method of generating MIPs.

FIG. 2A illustrates a schematic representation of generating MIPs to a macromolecule associated with a microorganism.

FIG. 2B illustrates a schematic representation of detecting a microorganism utilizing MIPs of the present invention.

FIG. 3A shows the chain view of SEQ ID NO:4 within the crystal structure of PBP2A.

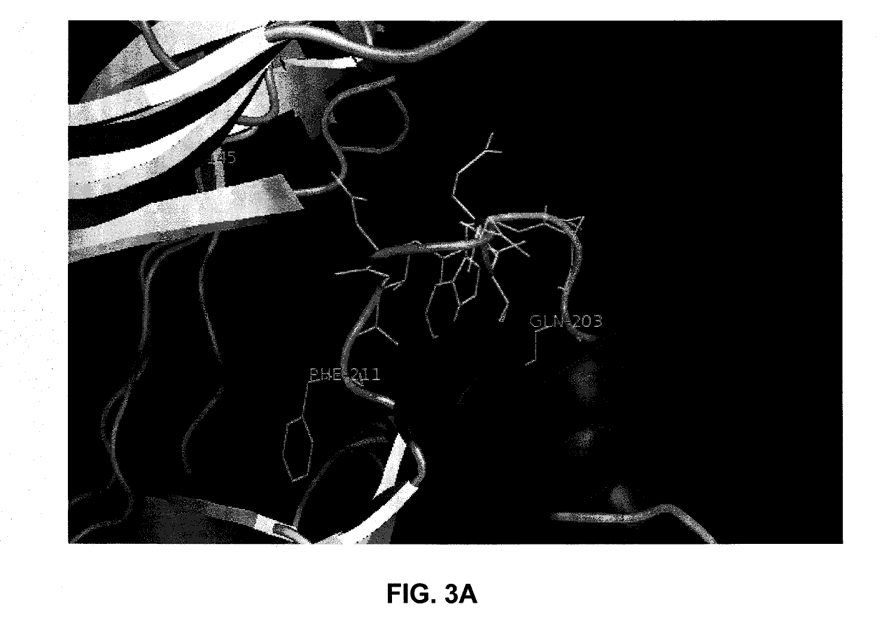

FIG. 3B shows the surface view of SEQ ID NO:4 within the crystal structure of PBP2A.

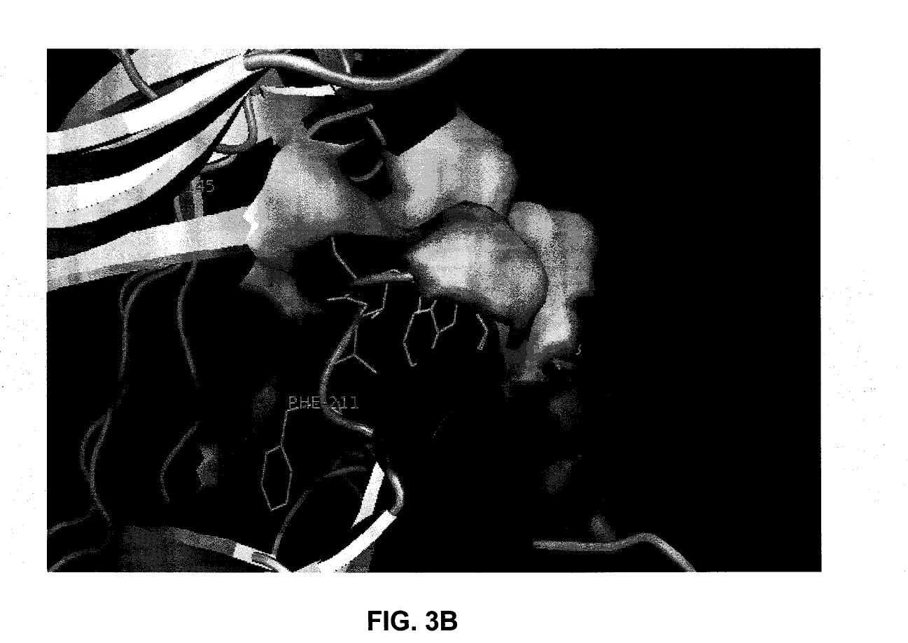

FIG. 4A shows the chain view of SEQ ID NO:5 within the crystal structure of PBP2A.

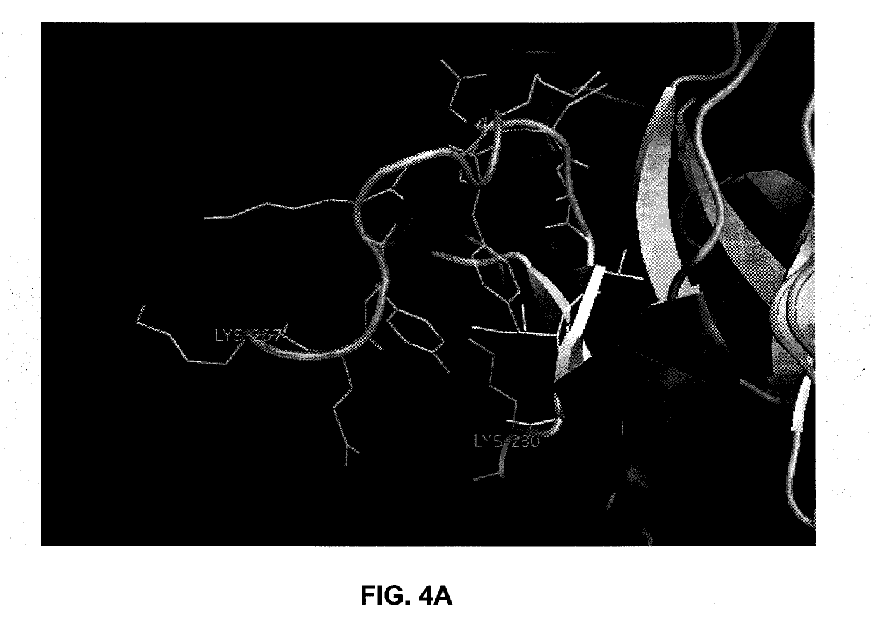

FIG. 4B shows the surface view of SEQ ID NO:5 within the crystal structure of PBP2A.

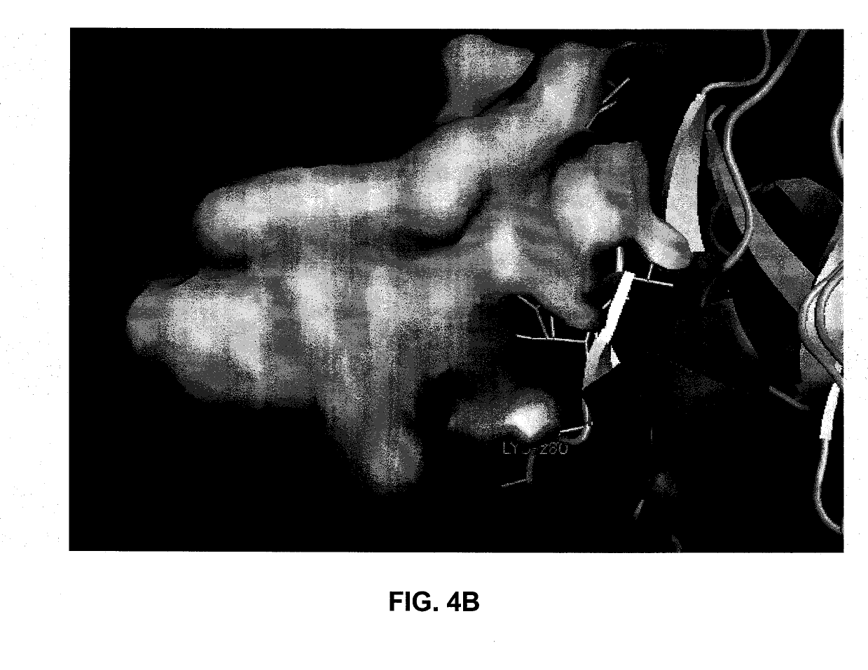

FIG. 5A shows the chain view of SEQ ID NO:8 within the crystal structure of PBP2A.

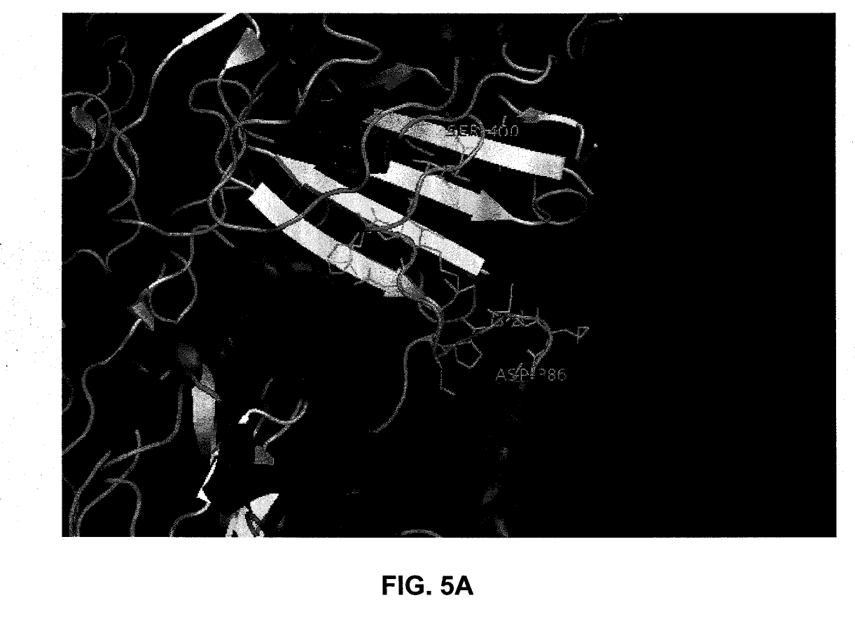

FIG. 5B shows the surface view of SEQ ID NO:8 within the crystal structure of PBP2A.

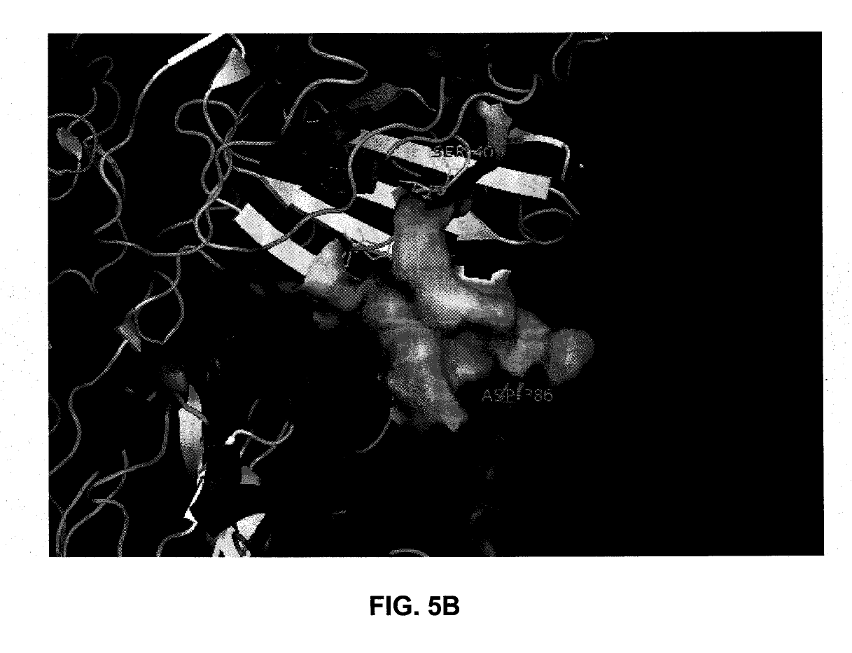

FIG. 6A shows the chain view of SEQ ID NO:12 within the crystal structure of PBP2A.

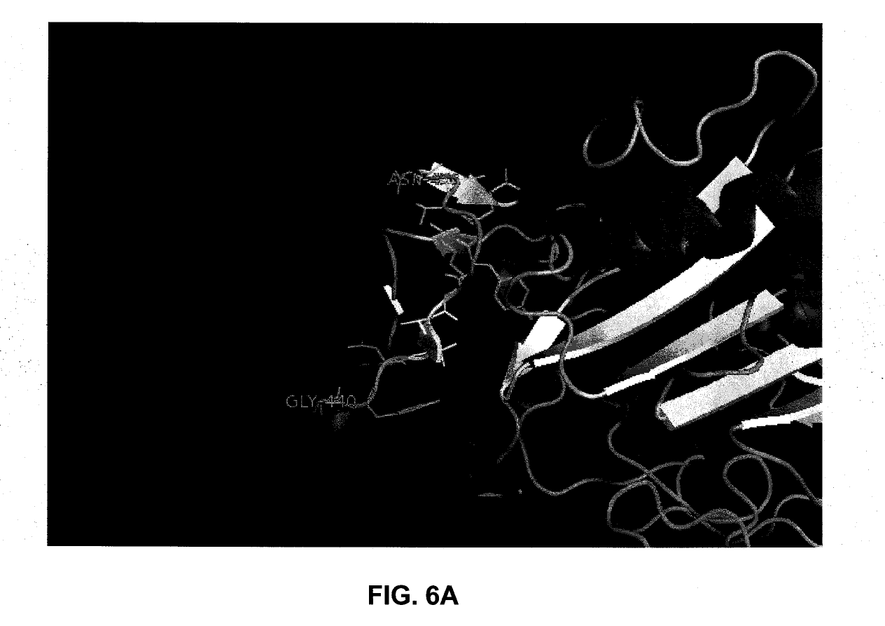

FIG. 6B shows the surface view of SEQ ID NO:12 within the crystal structure of PBP2A.

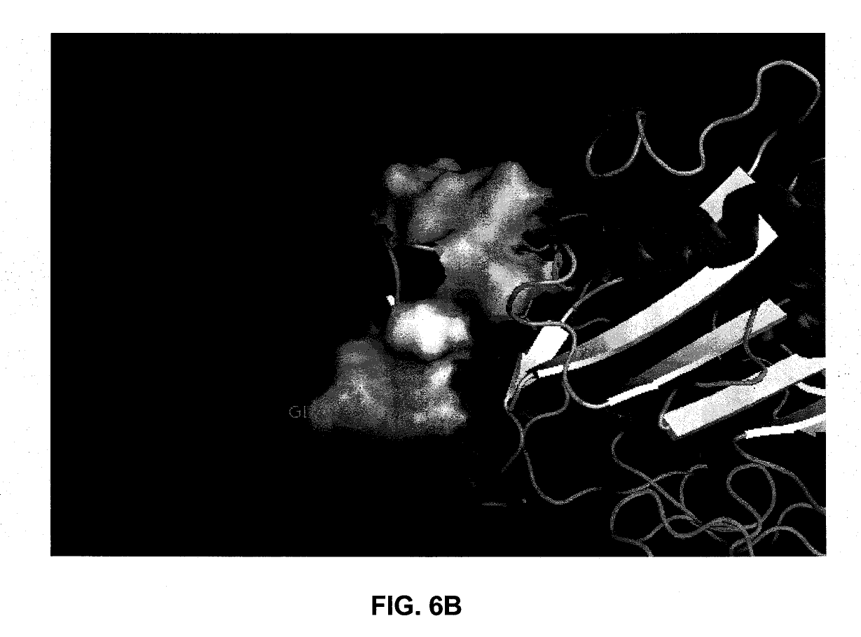

FIG. 7A shows the chain view of SEQ ID NO:13 within the crystal structure of PBP2A.

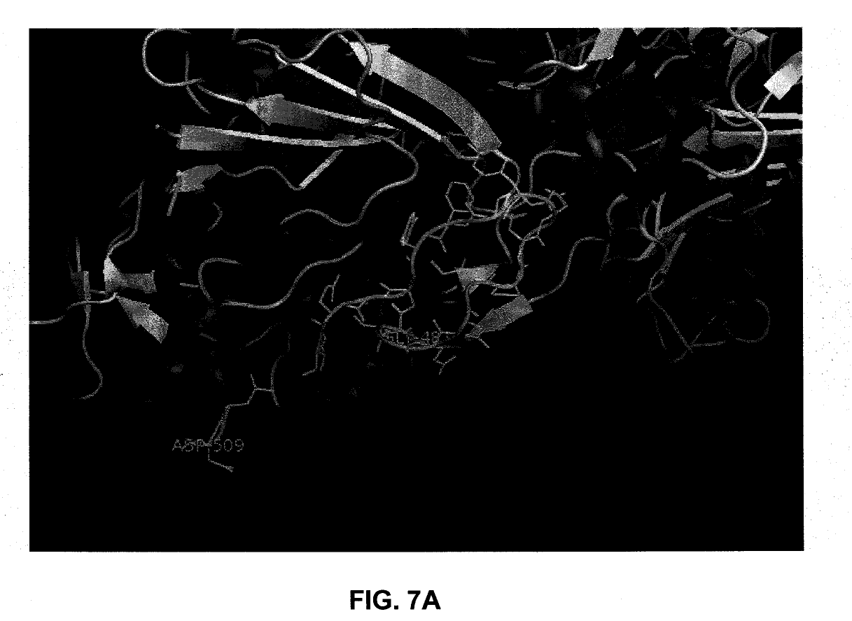

FIG. 7B shows the surface view of SEQ ID NO:13 within the crystal structure of PBP2A.

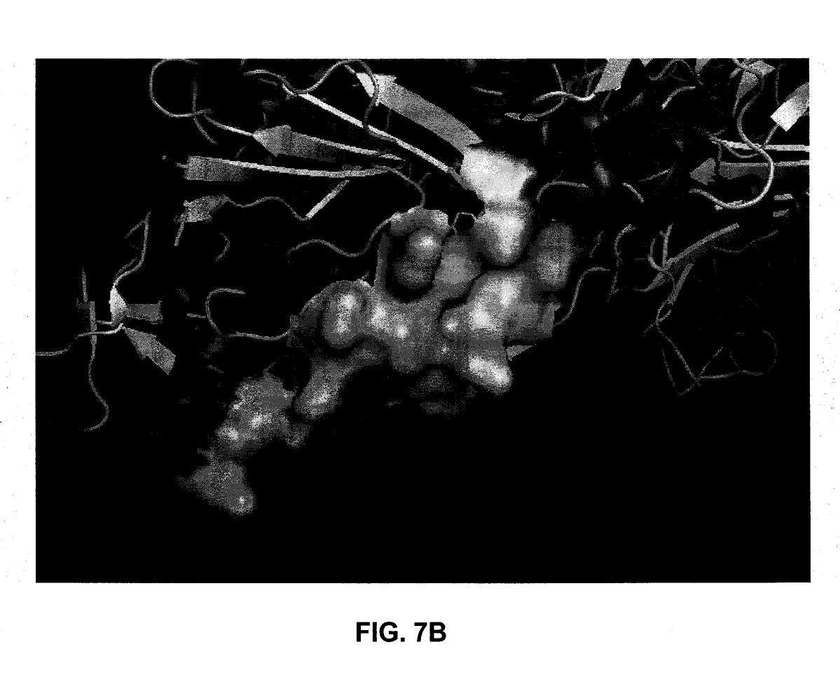

### DETAILED DESCRIPTION OF THE INVENTION

The invention described herein provides molecularly imprinted polymers (MIPs) capable of binding to a microorganism, and methods for detecting microorganisms utilizing such MIPs. Additionally, the invention provides methods and kits for detecting, identifying and/or quantifying microorganisms in biological samples, including, but not limited to, tissues, tissue extracts, and biological fluids. MIPs of the invention may be used to identify and/or quantify microorganisms on target surfaces and in liquid samples. MIPs of the invention may be used anywhere detection of an organism is desired, for example in home, clinic, doctor's office, hospital bedside, factory, or field. The methods can be used for detecting microorganisms in a variety of biological, environmental and industrial samples without the need for complicated sample preparation procedures, and thus are also suitable for use by untrained personnel even in field conditions. Further, the invention provides methods for diagnosing an infection associated with a microorganism utilizing MIPs.

The MIPs of the invention can be prepared in accordance with any technique known to those skilled in the art using a microorganism or a portion thereof as a template molecule. These methods include covalent imprinting (Wulff, 1982, Pure & Appl. Chem., 54, 2093-2102) whereby the monomers are covalently attached to the template and polymerized using a cross-linker. Subsequently, the template is cleaved from the polymer leaving template-specific binding cavities. Alternatively, a non-covalent imprinting method such as disclosed by U.S. Pat. No. 5,110,833, which portion is specifically incorporated herein by reference, may be used, whereby the monomers interact with the target molecule by non-covalent forces, and are then connected via a cross-linker to form target specific binding sites after removal of the target molecule. Combinations and variations on these methods may be used to construct thin molecularly imprinted films and membranes (Hong et al., 1998 Chem. Mater., 10, 1029-1033); imprinting on the surface of solid supports (Blanco-López, et. al., 2004, Anal. Bioanal. Chem., 378, 1922-1928; Sulitzky C. et al., 2002 Macromolecules, 35, 79-91); and microspheres (Ye et al., 2000, Macromolecules, 33, 8239 -8245). Further, methods for preparing MIPs are described in U.S. Pat. Nos. 4,406,792, 4,415,655, 4,532,232, 4,935,365, 4,960,762, 5,015,576, 5,208,155, 5,310,648, 5,321,102, 5,372,719, 5,786,428, 6,063,637, and 6,593,142, the portions of all of which disclosing the methods for preparing MIPs are specifically incorporated herein by reference.

For example, molecular imprinting involves making a polymer cast of a template molecule, wherein the template includes, but is not limited to, an epitope. The process of making the polymer cast involves dissolving the template molecule to be imprinted in a suitable solvent. Normally, an imprint composition comprising a co-monomer, cross-linking monomer and a polymerization initiator is added to the solvent comprising the desired template. Radiation (photochemical or ionizing) or thermal energy is then applied to the reaction mixture, comprising the imprint composition and the template, to drive the polymerization process, ultimately resulting in the formation of a solid polymer. The resulting polymer may be processed using conventional polymer processing technologies, assuming those processes do not alter the structure of the molecularly imprinted sites. The imprinted molecule is extracted using methods appropriate for dissociating the template molecule from the polymer. Details of template molecule dissociation from the polymer are dependent upon the nature of the chemical interaction between the target molecule and the polymer binding site. The polymer dissociated from the template molecule possesses binding sites optimized for the structural and electronic properties of such template molecule.

Preferably, the conditions under which the template molecule is imprinted are similar or identical to the conditions under which the macromolecule is to be captured. For instance, if the macromolecule is to be captured under denaturing conditions, then the template molecule should be imprinted under the same denaturing conditions. Similarly, if the macromolecule is to be captured under native conditions, then the template molecule should be imprinted under the same native conditions. Native and denaturing conditions are well-known to those of skill in the art. Many heat-sensitive compounds that can be used to make imprint compositions according to the invention are known in the art and include, by way of example and not limitation, hydrogels such as agarose, gelatins, moldable plastics, etc. Examples of other suitable hydrogels are described in U.S. Pat. No. 6,018,033, U.S. Pat. No. 5,277,915, U.S. Pat. No. 4,024,073, and U.S. Pat. No. 4,452,892, the portions of all of which that relate to imprinting are incorporated herein by reference.

Suitable non-limiting examples of monomers that can be used for preparing a polymer of the present invention include methylmethacrylate, other alkyl methacrylates, alkylacrylates, ally or aryl acrylates and methacrylates, cyanoacrylate, styrene, a-methyl styrene, vinyl esters, including vinyl acetate, vinyl chloride, methyl vinyl ketone, vinylidene chloride, acrylamide, methacrylamide, acrylonitrile, methacrylonitrile, ethylene glycol diacrylate, pentaerythritol dimethacrylate, pentaerythritol diacrylate, N,N′-methylenebisacrylamide, N,N′-ethylenebisacrylamide and N,N′-(1,2-dihydroxyethylene)bisacrylamide. Depending upon the choice of the monomers used, the polymer particles will have a variety of physical and mechanical properties, such as hydrophobicity/hydrophilicity, mechanical strength and ease or resistance to swelling in the presence of solvents.

The MIPs of the invention may take a variety of different forms. For example, they may be in the form of individual beads, disks, ellipses, or other regular or irregular shapes (collectively referred to as “beads”), or in the form of sheets. Each bead or sheet may comprise imprint cavities of a single template molecule, or they may comprise imprint cavities of two or more same or different template molecules. In one embodiment, the MIPs comprise imprint cavities of a plurality of different template molecules arranged in an array or other pattern such that the relative positions of the imprint cavities within the array or pattern correlate with their identities, i.e. the identities of the template molecules used to create them. Each position or address within the array may comprise an imprint cavity of a single template molecule, or imprint cavities of a plurality of different template molecules, depending upon the application. Moreover, the entire array or pattern may comprise unique imprint cavities, or may include redundancies, depending upon the application.

In one embodiment, the invention provides methods of manufacturing matrix materials comprising the imprint compositions. Such matrix materials include, but are not limited to, substances that are capable of undergoing a physical change from a fluid state to a semi-solid or solid state. In the fluid state, the particles of a matrix material move easily among themselves, and the material retains little or no definite form. A matrix material in the fluid state can be mixed with other compounds, including template molecules. In the semi-solid or solid state, the matrix materials are capable of forming and retaining cavities that complement the shape of template molecules. Examples of such matrix materials include heat sensitive hydrogels such as agarose, polymers such as acrylamide, and cross-linked polymers.

In one embodiment of the invention, the MIPs are capable of binding to a microorganism by way of binding to a macromolecule associated with the microorganism. Generally, MIPs can specifically bind to all or a portion of the macromolecules including, but not limited to, epitopes associated with the microorganism.

The template molecule of the invention described herein can be selected from the group consisting of all or a portion of the microorganism, a macromolecule or a portion thereof associated with the microorganism, and analogs thereof. The portion to which the template molecule corresponds may be an internal portion of the macromolecule and/or an external portion, and/or a terminal portion of the macromolecule. Alternatively, the portion may be a side-group or modification of the macromolecule, such as a polysaccharide group of a glycoprotein macromolecule, or a portion thereof.

Microorganisms that can be captured, detected, identified and/or quantified using the imprint compositions of the invention described herein include, but not limited to, any type of prokaryotes, eukaryotes, virus and prions. Examples of such microorgranisms include, but are not limited to, bacteria, algae, fungi, yeast, mycoplasmas, archeabacteria, mycobacteria, parasites and protozoa. In one embodiment, MIPs and methods of the invention may be used to detect Methicillin-Resistant S. aureus (MRSA). The target molecules can be selected from proteins, peptides, or glycoproteins that are unique to the organism. For example, African trypanosomiasis is caused by trypanosoma brucei a parasitic protist species that causes sleeping sickness in humans and nagana in animals in Africa. This obligate parasite has two hosts: an insect vector and mammalian host. Because of the large difference between these hosts the trypanosome undergoes complex changes during its life cycle to facilitate its survival in the insect gut and the mammalian bloodstream. It also features a unique and notable variable surface glycoprotein (VSG) coat in order to avoid the host's immune system. VSG genes are hugely variable at the sequence level. However, for them to fulfill their shielding function, different VSGs have strongly conserved structural features. VSGs are made up of a highly variable N terminal domain of around 300 to 350 amino acids, and a more conserved C terminal domain of around 100 amino acids. The C terminal domain forms a structural bundle of 4 alpha helices, while the N terminal domain forms a ‘halo’ around the helices. The tertiary structure of this halo is well conserved between different VSGs (in spite of wide variation in amino acid sequence) allowing different VSGs to form the physical barrier required to shield the trypanosome's surface. In one embodiment of the invention, MIPs are capable of binding to an epitope of the conserved halo and/or other conserved amino acid sequences.

Macromolecules associated with microorganisms that can be detected, identified and/or quantified using the imprint compositions of the invention include any type of macromolecule from which a template molecule can be designed and constructed according to the principles taught herein. Virtually any type of macromolecule can be detected, identified and/or quantified using the methods and compositions of the invention. Non-limiting examples include polysaccharides, proteins, glycoproteins, peptidoglycans, lipoproteins, peptides, polypeptides, and polynucleotides, and other macromolecular targets that will be apparent to those of skill in the art.

In general, the structural units of the macromolecule to which the template molecule corresponds are contiguous within the primary structure of the macromolecule. If one of skill in the art can identify a terminus or termini in the primary structure of the macromolecule, then a preferred template molecule corresponds to a template that includes a terminus of the macromolecule. Alternatively, the portion of the macromolecule to which the template molecule corresponds can be expressed in size as a fraction of the size of the entire macromolecule. For example, template molecules can correspond to a portion of the macromolecule that consists of from 1% to 5%, from 1 to 10%, from 1 to 15%, from 1 to 20%, from 1 to 25%, from 1 to 30%, from 1 to 35%, from 1 to 40%, from 1 to 50%, from 1 to 60%, from 1 to 70%, from 1 to 80%, from 1 to 90%, from 1 to 95%, or from 1 to 99% of the structure of the entire macromolecule. Preferably, template molecules have a primary structure that corresponds to a contiguous portion of the primary structure of the macromolecule.

If the macromolecule is a polypeptide, the template molecule can correspond to a portion of the polypeptide that consists of a sequence of amino acids selected from the primary sequence of the polypeptide or an analog thereof. For instance, the portion of the polypeptide can consist of a range of amino acids from the primary structure of the polypeptide consisting of from 1 to 50 amino acids, from 2 to 40 amino acids, from 3 to 30 amino acids, from 3 to 15 amino acids, from 3 to 10 amino acids, from 4 to 10 amino acids, from 4 to 9 amino acids, from 4 to 8 amino acids, from 4 to 7 amino acids, or from 5 to 7 amino acids. Preferred portions of the macromolecule are those that consist of a contiguous sequence of amino acids from the primary structure of the polypeptide.

When the macromolecule is a polynucleotide, the template molecule can be an oligonucleotide having a sequence of nucleotides selected from the primary sequence of the polynucleotide or an analog thereof. If the polynucleotide has n nucleotides, then the selected sequence of nucleotides can have a length from 1 to (n-1) nucleotides. Alternatively, the selected sequence can contain from 1 to 50 nucleotides, 2 to 40 nucleotides, 3 to 30 nucleotides, 3 to 15 nucleotides, 3 to 10 nucleotides, 4 to 10 nucleotides, 4 to 9 nucleotides, 4 to 8 nucleotides, 4 to 7 nucleotides, or 5 to 7 nucleotides. Preferably, the selected sequence is a contiguous sequence of nucleotides from the primary sequence of the polynucleotide.

It will be understood that as used herein, the expression “macromolecule” is not intended to place specific size limitations upon the molecules that may be identified with the MIPs of the methods described herein. Rather, macromolecules include molecules that comprise a plurality of structural moieties or analogs thereof such that a template molecule corresponding to at least one of the structural moieties can be used to prepare a molecular imprint capable of binding the macromolecule. In one embodiment of the invention, template molecules corresponding to at least two of the structural moieties can be used to prepare a molecular imprint capable of binding the macromolecule. In one embodiment of the invention, template molecules corresponding to at least three of the structural moieties can be used to prepare a molecular imprint capable of binding the macromolecule. In one embodiment of the invention, template molecules corresponding to at least four of the structural moieties can be used to prepare a molecular imprint capable of binding the macromolecule.

By “analog” is meant a molecule that differs from, but is structurally, functionally, and/or chemically related to the reference molecule. The analog may retain the essential properties, functions, or structures of the reference molecule. Most preferably, the analog retains at least one biological function of the reference molecule. Generally, differences are limited so that the structure or sequence of the reference molecule and the analog are similar overall. For example, a peptide analog and its reference peptide may differ in amino acid sequence by one or more substitutions, additions, and/or deletions, in any combination. Other examples of analogs include peptides with minor amino acid variations from the peptides exemplified herein. In particular, peptides containing conservative amino acid replacements, i.e., those that take place within a family of amino acids that are related in their side chains, constitute analogs. A substituted or inserted amino acid residue may or may not be one encoded by the genetic code. An analog of a peptide or polypeptide may be naturally occurring such as an allelic variant, or it may be a variant that is not known to occur naturally. Non-naturally occurring analogs of peptides may be made by direct synthesis, by modification, or by mutagenesis techniques.

The identities of the structural moieties that comprise macromolecules will depend upon the nature of the macromolecule, and may include regions of primary, secondary and/or tertiary structure of the macromolecule. For example, for polypeptide macromolecules the structural moieties may be the individual amino acids composing the polypeptide, or alternatively, if the polypeptide has several structural domains, the structural moieties may be the individual structural domains. For example, a polypeptide may be viewed as being composed of individual amino acids or structural domains as described above and/or saccharide or oligosaccharide structural moieties; a polynucleotide macromolecule may be viewed as being composed of individual nucleotide structural moieties. The macromolecules according to the invention may be derived from virtually any source. They may be obtained from natural sources such as biological samples or from synthetic sources.

Biofilm is an agglomeration of bacteria bound together by an exopolysaccharide (EPS) matrix composed of excreted proteins and/or polysaccharide polymers. During infection, the biofilm matrix acts as a safe haven, protecting bacterial cells from antibiotics, immune cells, and antimicrobial factors. In some embodiments, the MIPs are targeted to polysaccharides and/or proteins that are associated with the bacteria. Such polysaccharides and/or proteins may be part of the biofilm or the bacteria itself (e.g., many bacteria have a polysaccharide coating).

The composition of the biofilm for each species of bacteria is believed to be unique. For example, the chemicals, polysaccharides, proteins, etc. that make up the EPS is different for individual bacterium. Thus, MIPs targeted to a unique component of a bacterial biofilm may allow for species-specific detection of bacteria. The cell surface polysaccharides and/or proteins displayed by the bacteria may also be unique to each species of bacteria. For example, Staphylococcus aureus (S. aureus) has a unique cell surface fibronectin-binding protein (FnBP) for binding with fibronectin. Various epitopes of the S. aureus FnBP are disclosed, for example, by M. Huesca et al., Infection & Immunity, vol. 68:3, pp. 1156-1163 (March 2000) and Q. Sun et al., Infection & Immunity, vol. 65:2, pp. 537-543 (February 1997). MIPs targeted to such epitopes of S. aureus FnBP could be used for species-specific detection of S. aureus.

Cell surface proteins on bacteria also include a number of cell surface adhesion molecules that mediate adherence of the bacteria with a biofilm matrix. For example, FnBP is a member of the family of MSCRAMM proteins (microbial surface components recognizing adhesive matrix molecules) that are required for the initial attachment of bacteria to host, providing a critical step to establish infection. Other examples of members of this family include clumping factor A (C1fA) for S. aureus and the SdrG protein for S. epidermidis. In some embodiments, the MIPs may be targeted to macromolecules on endospores that are generated by bacteria. For example, Clostridrium difficile (C. difficile) is a species of bacteria known to generate endospores. An endospore is a tough, dormant structure produced by some bacteria to ensure the survival of a bacterium through periods of environmental stress. They are therefore resistant to ultraviolet and gamma radiation, desiccation, lysozyme, temperature, starvation, and chemical disinfectants. Endospores are commonly found in soil and water, where they may survive for long periods of time.

Accordingly, MIPs of the invention described herein can be used to detect and/or identify bacteria in a variety of environments, including bacteria existing in the planktonic mode, its biofilm, and/or its endospores. The planktonic form of the bacteria is the form of the bacterium when the bacterium is floating or swimming in a liquid medium. Various types of bacteria may be detected by the MIPs of the present invention. Such bacteria include S. aureus, methicillin-resistant S. aureus (MRSA), Enterococcus species, vancomycin-resistant Enterococcus (VRE), Clostridrium difficile (C. difficile), Pseudomonas aeruginosa (P. aeruginosa) and Escherichia coli (E. coli).

One embodiment of the invention provides for methods of manufacturing MIPs specific for MRSA utilizing all or portion of the MRSA bacteria and/or biofilm as a template molecule.

In one embodiment, methods of the invention encompass manufacturing MIPs specific for MRSA bacteria and/or biofilm comprising generating a template corresponding to all or portion of a macromolecule associated with the MRSA bacteria and/or biofilm and utilizing such template to manufacture MIPs.

In one embodiment, the invention provides for methods of identifying MRSA bacteria and/or biofilm, comprising utilizing MIPs that are capable of binding to all or a portion of PBP2a.

In another embodiment, the invention provides for methods of identifying MRSA bacteria and/or biofilm, comprising utilizing MIPs that are capable of binding to amino acid sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and/or fragments thereof.

Similarly, various microorganisms can be identified and/or detected utilizing a variety of macromolecules, as mentioned above, associated with said microorganisms as templates to generate MIPs. Non-limiting examples of the macromolecules include capsid or envelope proteins for detecting viruses, and surface coat glycoprotein for detecting parasites.

Alternatively, the MIPs of the invention are coupled with transduction elements such that a detectable signal is produced in response to binding of MIPs to said template or target molecule.

Suitable examples of transduction elements include, but not limited to, HABA [2(4′-hydroxyazo benzene)-benzoic acid], dyes, fluorescers, fluorescent dyes, radiolabels, magnetic particles, metallic particles, colored particles, metal sols, enzyme substrates, enzymes, chemiluminescers, photosensitizers and suspendable particles.

In some embodiments, the detectable signal may be a visible substance, such as a colored latex bead, or it may participate in a reaction by which a colored product is produced. The reaction product may be visible when viewed with the naked eye, or may be apparent, for example, when exposed to a specialized light source, such as ultraviolet light.

The concentration of template or the target molecule may be indicated by the amount of detectable signal associated with the transduction element.

Additionally, target detection by MIPs may be signaled in a variety of ways. In some cases, the detection signal may be visualized (e.g., luminescence or change in color). One such technique for providing a color change response using MIPs is explained in the document entitled “Molecularly Imprinted Polymer Sensor Aerosol” by George M. Murray, Ph.D., which is incorporated by reference herein in its entirety. A technique for using porphyrins with MIPs to cause a change in absorption/emission of electromagnetic radiation is explained in U.S. Pat. No. 6,872,786, the portion of which that describes the technique for using porphyrins with MIPs is incorporated herein by reference. Some techniques for producing luminescence using MIPs upon target detection are explained in U.S. Pat. No. 6,749,811, the portion of which that describes target detection using a MIP is incorporated herein by reference; A. L. Jenkins et al., Anal. Chem., vol. 71:2, pp. 373-378 (1999); and B. R. Arnold et al., Johns Hopkins APL Technical Digest, vol. 20:2, pp. 190-198 (1999). Any of the preceding techniques can be adapted for use with the MIPs of the present invention.

For example, MIPs in accordance with one aspect of the present invention can be prepared by (A) providing the reaction product of a polymerizable porphyrin derivative and a template molecule; (B) copolymerizing the reaction product of step (A) with monomer and crosslinking agent to form a polymer; and (C) removing the template molecule from the polymer to provide a molecularly imprinted polymer which exhibits selective binding affinity for the template molecule and undergoes a detectable change in absorption and/or emission of electromagnetic radiation when the target molecule binds thereto. The polymerization reaction mixture for preparation of MIP therefore constitutes the reaction product of step (A), one or more polymerizable monomers, an effective amount of one or more crosslinking agents to impart a sufficiently rigid structure to the polymer end-product, inert solvent, and a free radical or other appropriate initiator. Mixtures of monomers and crosslinking agents can be used in the polymerization method. The amounts of polymerizable porphyrin, monomer and crosslinking agents can vary broadly, depending on the specific nature/reactivities of the polymerizable porphyrin, monomer and crosslinking agent chosen as well as the specific sensor application and environment in which the polymer/sensor will be ultimately employed. The relative amounts of each reactant can be varied to achieve desired concentrations of porphyrin in the polymer support structure. The solvent, temperature and means of polymerization can be varied in order to obtain polymeric materials of optimal physical or chemical features, for example, porosity, stability, and hydrophilicity. The solvent can also be chosen based on its ability to solubilize all the various components of the reaction mixture.

Further, according to another embodiment of the invention described herein, the MIPs comprise lanthanide-containing polymeric structures that exhibit selective binding characteristics towards a target to be detected by a sensor device or kit of the invention described herein. The polymerization step comprises co-polymerizing a chelated lanthanide-template complex with one or more cross-linking monomers, and optionally, one or more additional matrix monomers to form a polymer structure. Any of a wide range of lanthanide metal salts capable of dissociating in solution to form a lanthanide ion, and combinations of two or more thereof, are suitable for use in the invention described herein. Examples of suitable lanthanide salts include, but are not limited to, halides, nitrates, perchlorates, and the like, of lanthanum (La), cerium (Ce), praseodymium (Pr), neodymium (Nd), promethium (Pm), samarium (Sm), europium (Eu), gadolinium (Gd), terbium (Th), dysprosium (Dy), holmium (Ho), erbium (Er), thulium (Tm), ytterbium (Yb), and lutetium (Lu). MIPs can be used as part of an optical sensor device to selectively capture the target, by associating such molecules with MIP lanthanide binding sites. Such MIPs act as a source of luminescence, which can be analyzed to determine the amount of target in the solution. Any of a wide range of suitable detectors can be used according to the invention described herein. Non-limiting examples of suitable detectors include a spectrophotometer, spectrometer (gas or mass), photomultiplier tube, monochromator equipped with a CCD camera, filters, the naked eye, combinations of two or more thereof, and the like.

The invention described herein additionally presents a method for forming a reliable chemical sensor platform based on site selectively tagged and templated molecularly imprinted polymers (SSTT-MIP). The SSTT-MIP strategy used in the present method provides a way to form a MIP having a templated site specific for an analyte and at which a reporter molecule can also be attached. In this way, analyte detection can be carried out with a higher efficiency in comparison to methodologies without any provision for such positioning. With this invention, measurement characteristics such as signal-to-background and signal-to-noise ratios are expected to be improved.

The invention also provides molecularly imprinted polymer platforms in which templated sites are formed for specific target molecules. In one embodiment, the polymer platforms can be provided wherein the templated sites have at least one reporter molecule bonded to a reactive group at the site. In one embodiment, the polymer platform comprises xerogels or aerogels. Methods to develop sensors for the detection of a wide variety of targets are described by Bright et al., in U.S. Publication No 2005/0227258, the portions of which that describe development of templated sites for specific target molecules, is incorporated herein by reference.

For example, the template is chosen such that it forms bonds with the polymer platform in excess of the number of those which will be formed by the bound target. The template can have at least one additional reactive group so as to be able to bind to the polymer matrix at an additional site relative to an intended target. In one embodiment, the number of reactive groups on the template which link it to the polymer platform, is at least one more than the number of reactive groups on the corresponding target. The removal of the template results in cavities within the polymer platform. Exposed at each templated site are reactive groups which are responsible for target recognition. However, as a consequence of the additional reactive group(s) mentioned above, when the template is cleaved from the cavity, the cavity bears one or more reactive groups in excess of the groups needed to bind the target. The extra group(s) is (are) used to bond with reporter molecules. Once the reporter molecule(s) is (are) bound at the templated site, the absorbance/luminescence from the target bound MIP can be measured and a change in UV, visible or IR absorbance/luminescence properties of the reporter (e.g., absorbance spectra, excitation and emission spectra, excited-state luminescence lifetime and/or luminescence polarization) indicates the presence of target at the templated site. The total change in absorbance/luminescence is generally proportional to the concentration of target molecule in the sample.

The MIPs of the invention can be utilized for diagnosing a subject infected with a microorganism, comprising contacting a biological sample obtained from said subject with one or more MIPs, and detecting and/or identifying the presence of said microorganism in said biological sample. The methods of diagnosis comprises measuring the level of all or a portion said microorganism, or all or a portion of macromolecule associated with said microorganism in a biological sample or biological fluid obtained from said patient.

The MIPs of the invention can also be utilized for determining the onset, progression, or regression of an infection associated with a microorganism in a subject, wherein a biological sample obtained from a subject is screened for all or a portion said microorganism, or all or a portion of macromolecule associated with said microorganism by contacting said biological sample with one or more MIPs.

As used herein, the phrase “biological sample” encompasses a variety of sample types obtained from a subject and useful in the procedure of the invention. Biological samples may include, but are not limited to, solid tissue samples, liquid tissue samples, biological fluids, aspirates, cells and cell fragments. Specific examples of biological samples include, but are not limited to, solid tissue samples obtained by surgical removal, pathology specimens, archived samples, or biopsy specimens, tissue cultures or cells derived therefrom and the progeny thereof, and sections or smears prepared from any of these sources. Non-limiting examples of biological samples include samples obtained from breast tissue, lymph nodes, and breast tumors. Biological samples also include any material derived from the body of a vertebrate animal, including, but not limited to, blood, cerebrospinal fluid, serum, plasma, urine, nipple aspirate, fine needle aspirate, tissue lavage such as ductal lavage, saliva, sputum, ascites fluid, liver, kidney, breast, bone, bone marrow, testes, brain, ovary, skin, lung, prostate, thyroid, pancreas, cervix, stomach, intestine, colorectal, brain, bladder, colon, nares, uterine, semen, lymph, vaginal pool, synovial fluid, spinal fluid, head and neck, nasopharynx tumors, amniotic fluid, breast milk, pulmonary sputum or surfactant, urine, fecal matter and other liquid samples of biologic origin.

The MIPs as described in the invention herein may be provided for use in a variety of media, sensors, devices, or products. For example, the MIPs of the present invention may be contained in a solution. As such, the solution can be sprayed onto an article to detect the target microorganism, for example, bacteria, its biofilm, and/or its endospore. In some embodiments, the solution may also contain an antimicrobial agent (e.g., antibiotics). The microorganisms may be detected on a variety of articles, such as environmental surfaces in hospitals, sports equipment, or medical devices. Some examples of MIP-based sensors are described in U.S. Pat. Nos 5,587,273, 6,680,210, 6,833,274, 6,967,103, 6,749,811 and 6,461,873; the portions of which that describe MIP-based sensors are specifically incorporated herein by reference.

In hospitals, the transferal of microorganisms from environmental surfaces to patients is largely via hand contact with the surface. Although hand hygiene is important to minimize the impact of transfer, cleaning and disinfecting environmental surfaces as appropriate is fundamental in reducing their potential contribution to the incidence of healthcare-associated infections. Thus, the MIP products of the invention described herein include, but are not limited to, a hand-wipe, impregnated with a solution of MIPs designed to detect a microorganism, that could aid in insuring that hands of clinical personnel are free of the microorganism by changing color if the microorganism is present on their hands, a spray of the solution that could be used in high touch areas (e.g., bed railing, door knobs, computer keyboards, etc.). Such MIP products could be used to demonstrate the effectiveness of cleaning efforts. Such MIP products could also be valuable in outbreak investigations. For example, by being able to distinguish between various microorganisms, for example, MRSA, VRE, E. coli. etc., it is possible to trace the path by which it is spread. Also, such MIP products can be used as an educational tool for training hospital staff.

The invention described herein also provides kits comprising MIPS as described above for specifically detecting, identifying and/or quantifying microorganisms. Such kits include, but are not limited to, dipstick, lateral-flow, flow-through, and migratory devices with one or more MIPs attached to a mobile or immobile solid phase material such as latex beads, glass fibers, glass beads, cellulose strips or nitrocellulose membranes, as described in U.S. Pat. Nos. 3,802,842, 3,915,639, 4,059,407, 4,373,932, 4,689,309, 4,703,017; 4,743,560, 4,770,853 5,073,484, 5,075,078; 5,096,837, 5,229,073, 5,354,692, 7,109,042, WO 88/08534 and WO 08/007359, the portions of which that describe the construction and function of above-mentioned kits and devices are incorporated herein by reference.

Dipstick devices, such as disclosed by Hochstrasser (U.S. Pat. No. 4,059,407) are designed to be immersed in a fluid biological sample and to a give a semi-quantitative estimation of the target in the fluid. Dipsticks are essentially lateral flow devices whose application method involves immersing the device in the liquid sample. Also of interest in the area of dipstick devices are U.S. Pat. Nos. 3,802,842, 3,915,639 and 4,689,309.

Lateral flow devices (see U.S. Pat. Nos. 5,075,078; 5,096,837; 5,354,692 and 5,229,073) generally comprise a porous matrix containing the relevant specific reagents, which is layered on a solid strip, such as plastic. Instead of vertically wicking the samples up the “dipstick,” the lateral flow format allows a sample to flow laterally across the porous, solid phase material by capillary action, across one or more reagents that interact with the target (if it is present in the sample). A visual signal (produced by colored beads, enzymatic reaction or other color-forming reactions) indicates the presence of the target.

In flow-through type devices, the applied test sample flows through a porous material, bringing the target in the sample in contact with the specific reagents contained in the porous material, eventually producing a visible signal on the porous material that provides an indication of the presence of target in the sample.

Visible detection of test results without the need to add external reagents is achieved in migration assay devices by incorporating reagents that have been coupled to colored labels (i.e., conjugates), thereby permitting visible detection of the assay results without addition of further substances. Such labels include, but are not limited to, gold sol particles, dye sol particles and dyed latex. In one embodiment, the diagnostic test devices of the invention described herein comprise two distinct pathways for the sample and the conjugated reagent.

One embodiment of the invention provides for a quantitative chromatographic test strip. The device consists of a strip that moves the sample solution by capillary action to zones in the strip containing the reagents that in the presence of the target produce a detectable signal.

Another embodiment of the invention discloses a chromatographic test strip comprising a solid support having two portions that permit capillary flow that is useful in a variety of immunoassays. The first portion includes a movable tracer and the second portion includes an immobilized binder capable of binding to the target.

Reagent-impregnated test strips have been used in various specific binding assays. The sample is applied to one portion of the test strip and migrates through the porous strip material, in some cases with the aid of an eluting solvent such as water. The sample advances into or through a detection zone where a specific binding reagent for the examined target is immobilized. The target present in the sample is then entrapped within the detection zone. The amount of bound target is determined usually by using labeled reagents incorporated in the test strip or applied subsequently. A variety of labels, such as radiolabels, chromophores, colored particles (gold, latex), enzymes, and fluorescent labels may be used in these assays. In most cases, the detecting binding agents are analyte-specific antibodies.

Further, in one embodiment, the invention is directed to a diagnostic device comprising a solid support capable of conveying a liquid sample therethrough, the sample being movable along or through the solid support in the path of liquid flow by capillary action. The support comprises: (a) a defined sample application area for applying the sample to the device and bringing it in contact with the solid support; (b) a defined MIP-conjugate zone downstream of the sample application area comprising a target-specific MIP fixed to the solid support on the flow path of the sample. The MIP has target- specific binding sites saturated with a releasable target analog:reporter conjugate in a dry state. The affinity of target to the binding sites of the target-specific MIP is greater than the affinity of the target analog:reporter conjugate to the binding sites of the target-specific MIP. The MIP, when contacted with a liquid sample containing the target, is capable of binding the target and displacing the target analog:reporter conjugate in an amount directly proportional to the concentration of the specific target, causing the displaced target analog:reporter conjugate to flow downstream in the path of liquid flow; (c) a defined results zone comprising a target analog:reporter conjugate binding element fixed to the solid support on the flow path of the sample downstream of the MIP-conjugate zone. The reporter conjugate binding element is capable of binding the target analog: reporter conjugate displaced from the MIP-conjugate zone when a liquid sample containing the target flows in the flow path zone for providing a detectable signal that indicates the presence or concentration of the target in a sample. Optionally, the solid support further comprises a reference zone for establishing a reference point in determining the presence or semi-quantification of an target in the tested sample, wherein the reference zone is not capable of capturing by specific binding any compound in said sample. The support may also optionally include a positive control zone comprising means for generating a positive control confirming the proper flow and binding of the target analog:reporter conjugate to the results zone to thereby determine that a test is working. Additionally, the support may optionally include an absorbent zone comprising a pad of absorbent material in fluid communication with the solid support when the pad and solid support are wet, the pad having sufficient porosity and capacity to absorb excess liquid.

In one embodiment, a sensor device as described herein comprises a molecularly imprinted polymer, containing a chelated lanthanide, capable of binding the target to be detected, and which has operatively associated therewith: a light source for generating excitation energy for the chelated lanthanide of the molecularly imprinted polymer, wherein at least a portion of the excitation energy is absorbed molecularly imprinted polymer; and a detector for detecting luminescent energy generated by the chelated lanthanide upon excitation.

It is to be understood that application of the teachings of the present invention to a specific problem or environment will be within the capability of one having ordinary skill in the art in light of teachings contained herein. The present invention is more fully illustrated by the following non-limiting examples.

### EXAMPLES

### Example 1

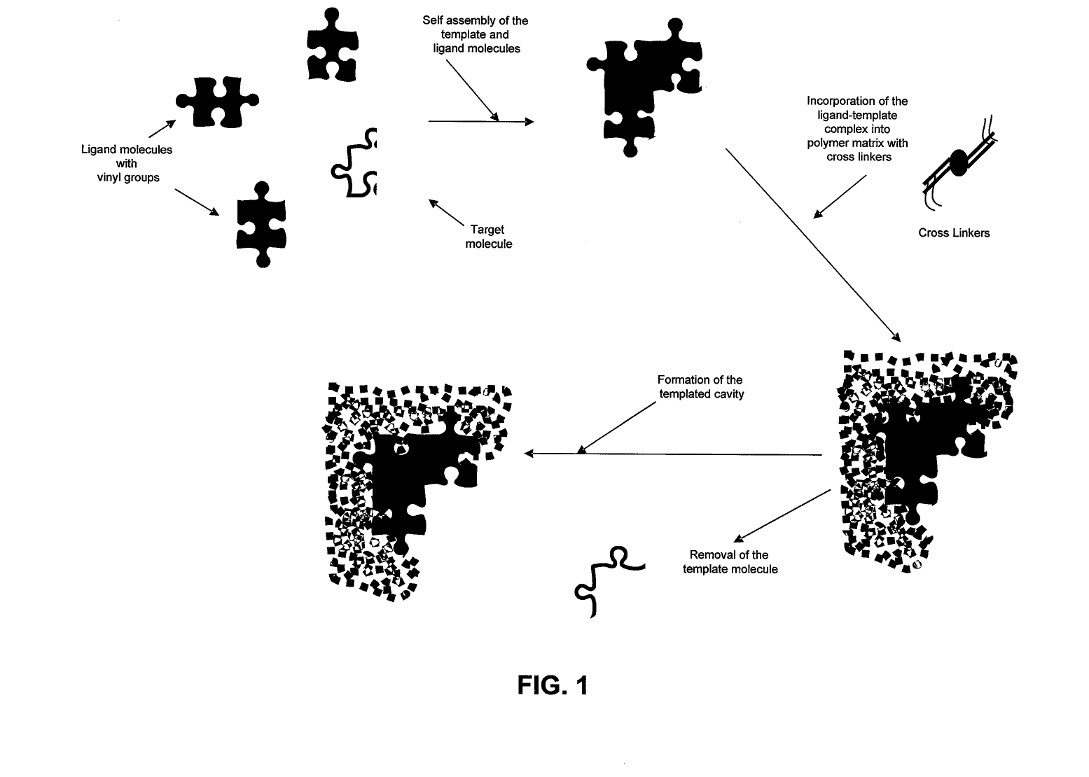

As illustrated in FIG. 1, MIPs of the invention described herein can be manufactured by generating a template of a portion of the target molecule, and polymerizing functional monomers in the presence of such template. The functional monomers can bind to active sites on the template molecule and then polymerize in the presence of excess of cross-linking agents. While the polymerization can be effected in the presence of the template molecules, subsequent removal of the latter can leave behind cavities that have the shape and an arrangement of the functional group that is complementary to that of the template molecules. Thus the resulting MIP can exhibit the ability to rebind the template molecule tightly and selectivity.

### Example 2

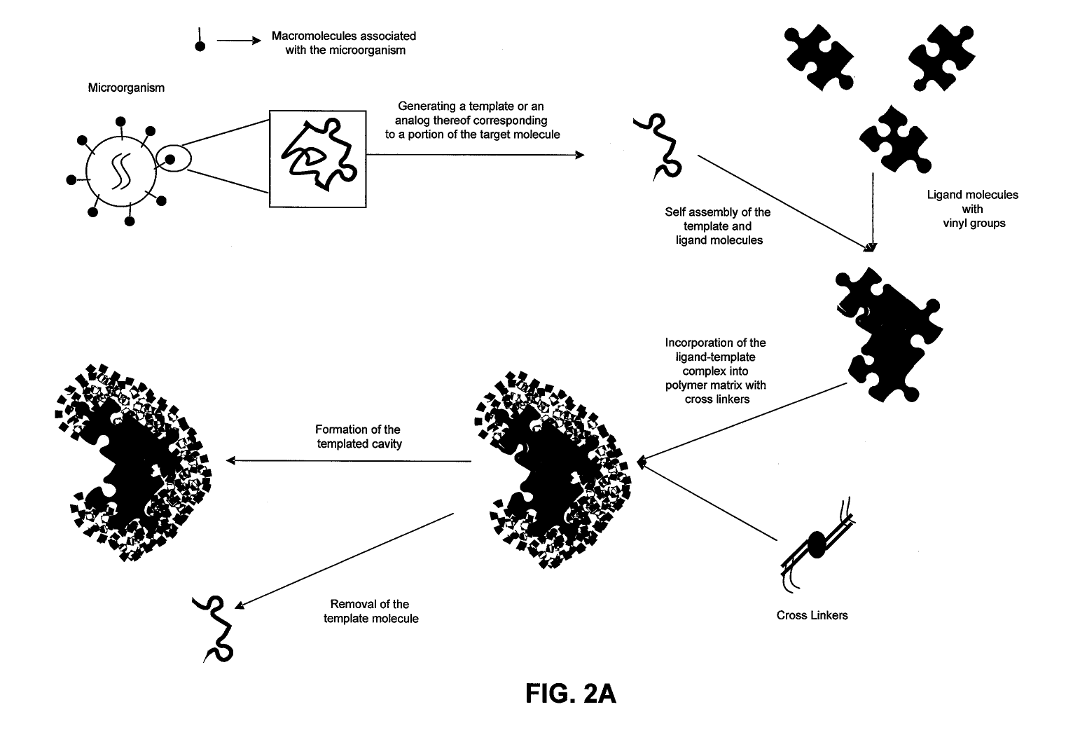

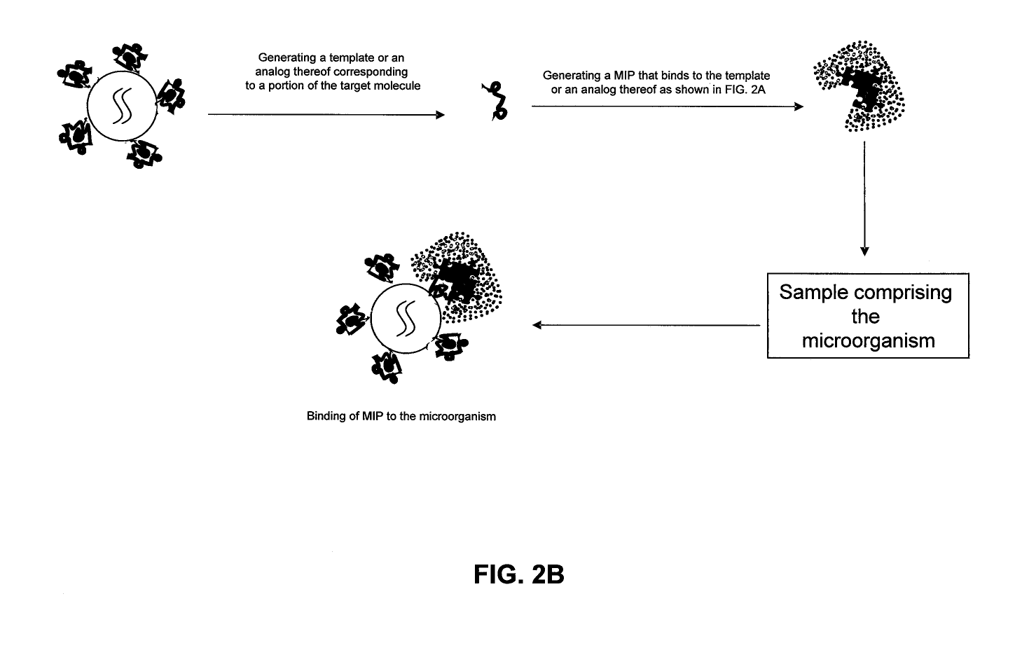

FIGS. 2A and 2B illustrate a schematic representation of detecting a bacteria utilizing MIPs of the present invention. Upon identification of a unique macromolecule associated with the bacteria, a template of a portion of the macromolecule comprising one or more epitopes, can be generated. Functional monomers can be polymerized in the presence of the template molecule such that the monomers bind to active sites on the template molecule, which can further be polymerized in the presence of excess of cross-linking agents. Subsequent removal of the template molecule (FIG. 2A) can leave behind cavities that have the shape and an arrangement of the functional group that is complimentary to that of the portion of the macromolecule that is unique to the microorganism. The resulting imprinted polymer can thus exhibit the ability to bind the portion of the macromolecule associated with the microorganism tightly and selectivity, and identify the microorganism (FIG. 2B).

### Example 3

Detection of Methicillin-Resistant S. aureus (MRSA) utilizing MIPs

One embodiment of the invention comprises detecting MRSA utilizing MIPS generated to bind to PBP2A. Particularly, MIPs can be generated utilizing epitopes amino acid sequences corresponding to various within PBP2A as template molecules. For example, template molecules can be designed comprising the amino acid sequences (please see table 1 below) MKKIKIVPLILIVVVVGFGIYFYAS (SEQ ID NO:1); KKIKIVPL (SEQ ID NO:2); KIKIVPLI (SEQ ID NO:3); QNWVQDDTF (SEQ ID NO:4); KEYKGYKDDAVIGK (SEQ ID NO:5); EYKGYKDD (SEQ ID NO:6); YKGYKDDA (SEQ ID NO:7); DKKEPLLNKFQITTS (SEQ ID NO:8); KEPLLNKF (SEQ ID NO:9); EPLLNKFQ (SEQ ID NO:10); PLLNKFQI (SEQ ID NO:11); GYNVTRYEVVN (SEQ ID NO:12); GVGEDIPSDYPFYNAQILD (SEQ ID NO:13); DYPFYNAQ (SEQ ID NO: 14) and/or fragments thereof, which are unique to provide a surface imprint with specificity for PBP2A. MIPs generated utilizing such amino acid sequences as templates can be used to detect and identify MRSA bacteria and/or biofilm.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

Modifications may be made without departing from the basic spirit of the present invention. Accordingly, it will be appreciated by those skilled in the art that within the scope of the appended claims, the invention may be practiced other than has been specifically described herein

TABLE 1

| | |
|---|---|
| SEQ ID NO: 1 | MKKIKIVPLILIVVVVGFGIYFYAS |
| SEQ ID NO: 2 | KKIKIVPL |
| SEQ ID NO: 3 | KIKLVPLI |
| SEQ ID NO: 4 | QNWVQDDTF |
| SEQ ID NO: 5 | KEYKGYKDDAVIGK |
| SEQ ID NO: 6 | EYKGYKDD |
| SEQ ID NO: 7 | YKGYKDDA |
| SEQ ID NO: 8 | DKKEPLLNKFQITTS |
| SEQ ID NO: 9 | KEPLLNKF |
| SEQ ID NO: 10 | EPLLNKFQ |
| SEQ ID NO: 11 | PLLNKFQI |
| SEQ ID NO: 12 | GYNVTRYEVVN |
| SEQ ID NO: 13 | GVGEDIPSDYPFYNAQILD |
| SEQ ID NO: 14 | DYPFYNAQ |

What is claimed is:

1. A molecularly imprinted polymer (MIP) capable of binding to all or a portion of a macromolecule associated with Methicillin-Resistant S. aureus (MRSA).

2. The MIP of claim 1, wherein said macromolecule is penicillin binding protein 2a (PBP2a).

3. The MIP of claim 1, wherein said portion of a macromolecule is selected from the group consisting SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and a fragment thereof.

4. The MIP of claim 1, wherein the MIP comprises a transduction element such that a measurable signal is produced in response to binding of MRSA to said MIP.

5. The MIP of claim 2, wherein the binding of PBP2a to said MIP produces a detection signal.

6. The MIP of claim 5, wherein the binding of said portion of a macromolecule selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and a fragment thereof produces a detection signal.

7. A method of detecting MRSA in a biological sample, comprising:
contacting said biological sample with a MIP capable of binding to all or a portion of a macromolecule associated with Methicillin-Resistant S. aureus (MRSA).

8. The method of claim 7, wherein said macromolecule is penicillin binding protein 2a (PBP2a).

9. The method of claim 7, wherein said portion of a macromolecule is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and a fragment thereof.

10. A method of detecting MRSA biofilm in a biological sample, comprising:
contacting said biological sample with a MIP capable of binding to all or a portion of a macromolecule associated with Methicillin-Resistant S. aureus (MRSA) or the biofilm.

11. The method of claim 10, wherein said macromolecule is penicillin binding protein 2a (PBP2a).

12. The method of claim 10, wherein said portion of a macromolecule is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and a fragment thereof.

13. A method of diagnosing a patient infected with MRSA, comprising:
contacting a biological sample obtained from said patient with one or more MIPs and detecting the presence of MRSA in said biological sample, wherein said one or more MIPs are capable of binding to all or a portion of a macromolecule associated with MRSA.

14. The method of claim 13, wherein said macromolecule is penicillin binding protein 2a (PBP2a).

15. The method of claim 13, wherein said portion of a macromolecule is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14 and a fragment thereof.

16. A molecularly imprinted polymer (MIP) capable of binding to a microorganism.

17. The MIP of claim 16, wherein said microorganism is selected from the group consisting of prokaryotes, eukaryotes, virus and prions.

18. The MIP of claim 16, wherein the MIP is capable of binding to all or a portion of a macromolecule unique to said microorganism.

19. The MIP of claim 18, capable of specifically binding to an epitope of said macromolecule.

20. The MIP of claim 18, wherein said macromolecule is selected from the group consisting of exopolysaccharides, polysaccharides, proteins, glycoproteins, peptidoglycans, lipoproteins, peptides, polypeptides, and polynucleotides.

21. The MIP of claim 16, wherein the MIP comprises one or more transduction elements such that a measurable signal is produced in response to binding of MIPs to said microorganism.

22. The MIP of claim 21, wherein said signal is selected from the group consisting of calorimetric, fluorescence, radioactive and enzymatic.

23. A method of detecting or identifying a microorganism, comprising:
contacting one or more molecularly imprinted polymers (MIPs) with said microorganism.

24. The method of claim 23, wherein said microorganism is selected from the group consisting of prokaryotes, eukaryotes, virus and prions.

25. The method of claim 23, comprising identifying all or a portion of a macromolecule unique to said microorganism.

26. The method of claim 25, wherein said MIPs specifically bind to an epitope of said macromolecule.

27. The method of claim 25, wherein said macromolecule is selected from the group consisting of exopolysaccharide, polysaccharides, proteins, glycoproteins, peptidoglycans, lipoproteins, peptides, polypeptides, and polynucleotides.

28. The method of claim 23, wherein said MIPs comprise one or more transduction elements such that a measurable signal is produced in response to binding of MIPs to said microorganism.

29. The method of claim 28, wherein said signal is selected from the group consisting of calorimetric, fluorescence, radioactive and enzymatic.

30. A method of diagnosing a patient infected with a microorganism, comprising:
contacting a biological sample obtained from said patient with one or more molecularly imprinted polymers (MIPs) and detecting the presence of said microorganism in said biological sample.

31. The method of claim 30, wherein said microorganism is selected from the group consisting of prokaryotes, eukaryotes, virus and prions.

32. The method of claim 30, comprising identifying all or a portion of a macromolecule unique to said microorganism.

33. The method of claim 32, wherein said MIPs specifically bind to an epitope of said macromolecule.

34. The method of claim 32, wherein said macromolecule is selected from the group consisting of exopolysaccharides, polysaccharides, proteins, glycoproteins, peptidoglycans, lipoproteins, peptides, polypeptides, and polynucleotides.

35. The method of claim 30, wherein the biological sample is selected from the group consisting of a biological fluid, tissue extract or tissues.

36. The method of claim 35, wherein the biological fluid is selected from the group consisting of blood, cerebrospinal fluid, serum, plasma, urine, nipple aspirate, fine needle aspirate, tissue lavage, saliva, sputum, ascites fluid, semen, lymph, vaginal pool, synovial fluid, spinal fluid, amniotic fluid, breast milk, pulmonary sputum or surfactant, urine, fecal matter, fluids collected from any of liver, kidney, breast, bone, bone marrow, testes, brain, ovary, skin, lung, prostate, thyroid, pancreas, cervix, stomach, intestine, colorectal, brain, bladder, colon, nares and uterine, head and neck, nasopharynx tumors, and other liquid samples of biologic origin.

37. The method of claim 30, wherein said MIPs comprise one or more transduction elements such that a measurable signal is produced in response to binding of MIPs to said microorganism.

38. The method of claim 37, wherein said signal is selected from the group consisting of calorimetric, fluorescence, radioactive and enzymatic.

39. A kit comprising MIPs for detecting or identifying a microorganism.

40. The kit of claim 39, wherein said microorganism is selected from the group consisting of prokaryotes, eukaryotes, virus and prions.

41. The kit of claim 39, wherein said MIPs are capable of identifying all or a portion of a macromolecule unique to said microorganism.

42. The kit of claim 41, wherein said MIPs specifically bind to an epitope of said macromolecule.

43. The kit of claim 39, wherein said MIPs comprise one or more with transduction elements such that a measurable signal is produced in response to binding of MIPs to said microorganism.

44. The method of claim 43, wherein said signal is selected from the group consisting of calorimetric, fluorescence, radioactive and enzymatic.


SEQUENCE LISTING (14 sequences)

SEQ ID NO: 1 - protein, 25 aa, Staphylococcus aureus
MKKIKIVPLILIVVVVGFGIYFYAS

SEQ ID NO: 2 - protein, 8 aa, Staphylococcus aureus
KKIKIVPL

SEQ ID NO: 3 - protein, 8 aa, Staphylococcus aureus
KIKIVPLI

SEQ ID NO: 4 - protein, 9 aa, Staphylococcus aureus
QNWVQDDTF

SEQ ID NO: 5 - protein, 14 aa, Staphylococcus aureus
KEYKGYKDDAVIGK

SEQ ID NO: 6 - protein, 8 aa, Staphylococcus aureus
EYKGYKDD

SEQ ID NO: 7 - protein, 8 aa, Staphylococcus aureus
YKGYKDDA

SEQ ID NO: 8 - protein, 15 aa, Staphylococcus aureus
DKKEPLLNKFQITTS

SEQ ID NO: 9 - protein, 8 aa, Staphylococcus aureus
KEPLLNKF

SEQ ID NO: 10 - protein, 8 aa, Staphylococcus aureus
EPLLNKFQ

SEQ ID NO: 11 - protein, 8 aa, Staphylococcus aureus
PLLNKFQI

SEQ ID NO: 12 - protein, 11 aa, Staphylococcus aureus
GYNVTRYEVVN

SEQ ID NO: 13 - protein, 19 aa, Staphylococcus aureus
GVGEDIPSDYPFYNAQILD

SEQ ID NO: 14 - protein, 8 aa, Staphylococcus aureus
DYPFYNAQ